(19) United States
(12) Patent Application Publication
(10) Pub. No.: US 2026/0335502 A1
(43) Pub. Date: Oct. 29, 2026

(54) UNIFORM THIN SPUN LAYER FOR COVERED MEDICAL APPLIANCES

(71) Applicant: Merit Medical Systems, Inc., South Jordan, UT (US)

(72) Inventors: John Hall, Bountiful, UT (US); Wayne Mower, Bountiful, UT (US); Darrell Skousen, Lehi, UT (US)

(21) Appl. No.: 19/464,293

(22) Filed: Jan. 29, 2026

Related U.S. Application Data
(60) Provisional application No. 63/752,467, filed on Jan. 31, 2025.
(60) Provisional application No. 63/915,580, filed on Nov. 11, 2025.

(51) Int. Cl. A61L 27/16

(52) U.S. Cl. CPC A61L 27/16; A61L 2420/02

(57) ABSTRACT

A medical appliance or prosthesis may comprise one or more layers of rotational spun nanofibers, including rotational spun polymers. The rotational spun material may comprise layers including layers of polytetrafluoroethylene (PTFE). Rotational spun nanofiber mats of certain thicknesses and uniformity may permit tissue ingrowth into or attachment to the prosthesis.

### RELATED CASES

This application claims priority to U.S. Provisional Application No. 63/752,467, filed on Jan. 31, 2025, and titled “UNIFORM THIN SPUN LAYER FOR COVERED MEDICAL APPLIANCES,” and to U.S. Provisional Application No. 63/915,580, filed on Nov. 11, 2025, and titled “UNIFORM THIN SPUN LAYER FOR COVERED MEDICAL APPLIANCES,” which are hereby incorporated by reference in their entirety.

### TECHNICAL FIELD

The present disclosure relates generally to medical devices. More specifically, the present disclosure relates to medical appliances or other prostheses, particularly those made of, constructed from, covered or coated with rotational spun materials including polymers such as polytetrafluoroethylene (PTFE).

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A is a perspective view of a rotational spinning apparatus, according to embodiments described herein.

FIG. 1B is a top view of the rotational spinning apparatus of FIG. 1A, according to embodiments described herein.

FIG. 2A is a perspective view of another embodiment of a rotational spinning apparatus, according to embodiments described herein.

FIG. 2B is a top view of the rotational spinning apparatus of FIG. 2A, according to embodiments described herein.

FIG. 3A is a perspective view of a covered stent, according to embodiments described herein.

FIG. 3B is a cross-sectional view of the covered stent of FIG. 3A taken through line 3B-3B, according to embodiments described herein.

FIG. 4A is a perspective view of a rotational spun covering on a mandrel.

FIG. 4B is a perspective view of the covering of FIG. 4A partially removed from the mandrel, according to embodiments described herein.

FIG. 4C is a perspective view of the covering of FIG. 4A repositioned on the mandrel, according to embodiments described herein.

FIG. 4D is a perspective view of a scaffolding structure wound around the covering and mandrel of FIG. 4C, according to embodiments described herein.

FIG. 4E is a perspective view of the scaffolding structure of FIG. 4D with a second rotational spun covering, according to embodiments described herein.

FIG. 5 is a perspective view of a covered stent including cuffs, according to embodiments described herein.

FIG. 6A is a cross-sectional view of a base tube on a mandrel, according to embodiments described herein.

FIG. 6B is a cross-sectional view of a base tube with a laser micrometer measuring the thickness of the base tube to determine if portions of the base tube have a thickness outside a range of predetermined thickness, according to embodiments described herein.

FIG. 6C is a cross-sectional view of a base tube with portions of the base tube outside of a range of predetermined thickness removed, according to embodiments described herein.

FIG. 6D is a cross-sectional view of a base tube with a first end and a second end that are outside a range of predetermined thickness are bunched up, according to embodiments described herein.

### DETAILED DESCRIPTION

Medical appliances may be deployed in various body lumens for a variety of purposes. Stents may be deployed, for example, in the central venous system or a variety of therapeutic purposes including the treatment of occlusions within the lumens of that system. The current disclosure may be applicable to stents or other medical appliances designed for the central venous (“CV”) system, peripheral vascular (“PV”) stents, abdominal aortic aneurism (“AAA”) stents, bronchial stents, esophageal stents, biliary stents, coronary stents, gastrointestinal stents, neuro stents, thoracic aortic endographs, or any other stent or stent graft. Further, the present disclosure may be equally applicable to other prosthesis such as grafts. Any medical appliance comprised of materials herein described may be configured for use or implantation within various areas of the body, including vascular, cranial, thoracic, pulmonary, esophageal, abdominal, or ocular application. Examples of medical appliances within the scope of this disclosure include, but are not limited to, stents, vascular grafts, stent grafts, cardiovascular patches, reconstructive tissue patches, hernia patches, general surgical patches, heart valves, sutures, dental reconstructive tissues, medical device coverings and coatings, gastrointestinal devices, blood filters, artificial organs, ocular implants, and pulmonary devices, including pulmonary stents. For convenience, many of the specific examples included below reference stents. Notwithstanding any of the particular medical appliances referenced in the examples or disclosure below, the disclosure and examples may apply analogously to any prostheses or other medical appliance.

As used herein, the term stent refers to a medical appliance configured for use within a bodily structure, such as within a body lumen. A stent may comprise a scaffolding or support structure, such as a frame, and/or a covering. Thus, as used herein, “stent” refers to both covered and uncovered scaffolding structures.

It will be readily understood that the components of the embodiments as generally described and illustrated in the Figures herein could be arranged and designed in a wide variety of different configurations. Thus, the following more detailed description of various embodiments, as represented in the Figures, is not intended to limit the scope of the disclosure, but is merely representative of various embodiments. While the various aspects of the embodiments are presented in drawings, the drawings are not necessarily drawn to scale unless specifically indicated.

The phrases “connected to,” “coupled to,” and “in communication with” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “proximal” and “distal” are used herein to refer to opposite locations on a stent or another medical appliance. The proximal end of an appliance is defined as the end closest to the practitioner when the appliance is disposed within a deployment device which is being used by the practitioner. The distal end is the end opposite the proximal end, along the longitudinal direction of the appliance, or the end furthest from the practitioner. It is understood that, as used in the art, these terms may have different meanings once the appliance is deployed (i.e., the “proximal” end may refer to the end closest to the head or heart of the patient depending on application). For consistency, as used herein, the ends labeled “proximal” and “distal” prior to deployment remain the same regardless of whether the appliance is deployed. The longitudinal direction of a stent is the direction along the axis of a generally tubular stent. In embodiments where a stent or another appliance is composed of a metal wire structure coupled to one or more layers of a film or sheet like components, such as a polymer layer, the metal structure is referred to as the “scaffolding” or “frame,” and the polymer layer as the “covering” or “coating.” The terms “covering” or “coating” may refer to a single layer of polymer, multiple layers of the same polymer, or layers comprising distinct polymers used in combination. Furthermore, as used herein, the terms “covering” and “coating” refer only to a layer or layers which are coupled to a portion of the scaffold; neither term requires that the entire scaffold be “covered” or “coated.” In other words, medical appliances wherein portion of the scaffold may be covered and a portion remain bare, are within the scope of this disclosure. Finally, any disclosure recited in connection with coverings or coatings may analogously be applied to medical devices comprising one or more “covering” layers with no associated frame or other structure. For example, a hernia patch comprising any of the materials described herein as “coatings” or “coverings” is within the scope of this disclosure regardless of whether the patch further comprising a frame or other structure.

Medical device coverings may comprise multilayered constructs, comprised of two or more layers which may be serially applied. Further, multilayered constructs may comprise nonhomogeneous layers, meaning adjacent layers have differing properties. Thus, as used herein, each layer of a multilayered construct may comprise a distinct layer, either due to the distinct application of the layers or due to differing properties between layers.

Additionally, as used herein, “tissue ingrowth” or “cellular penetration” refer to any presence or penetration of a biological or bodily material into a component of a medical appliance. For example, the presence of body tissues (e.g. collagen, cells, and so on) within an opening or pore of a layer or component of a medical appliance comprises tissue ingrowth into that component. Further, as used herein, “attachment” of tissue to a component of a medical appliance refers to any bonding or adherence of a tissue to the appliance, including indirect bonds. For example, tissue of some kind (e.g. collagen) may become attached to a stent covering (including attachment via tissue ingrowth) and another layer of biologic material (such as endothelial cells) may, in turn, adhere to the first tissue. In such instances, the second biologic material (endothelial cells in the example), and the tissue (collagen in the example) are “attached” to the stent covering.

Furthermore, through the present disclosure, certain fibrous materials (such as rotational spun materials) may be referred to as inhibiting or promoting certain biological responses. These relative terms are intended to reference the characteristics of the fibrous materials with respect to non-fibrous materials or coatings. Examples of non-fibrous coatings include non-fibrous PTFE sheets, other similarly formed polymers, and the like. Examples of fibrous coatings include rotational spun PTFE, electrospun PTFE, expanded PTFE, and other similarly formed polymers or materials. Examples of spun fibrous coatings include rotational spun PTFE, electrospun PTFE, and other similarly formed polymers or materials, and exclude expanded PTFE.

Lumens within the circulatory system are generally lined with a single layer (monolayer) of endothelial cells. This lining of endothelial cells makes up the endothelium. The endothelium acts as an interface between blood flowing through the lumens of the circulatory system and the inner walls of the lumens. The endothelium, among other functions, reduces or prevents turbulent blood flow within the lumen. The endothelium plays a role in many aspects of vascular biology, including atherosclerosis, creating a selective barrier around the lumen, blood clotting, inflammation, angiogenesis, vasoconstriction, and vasodilation.

A therapeutic medical appliance which includes a covering of porous or semi-porous material may permit the formation of an endothelial layer onto the porous surface of the blood contact side of the medical device. Formation of an endothelial layer on a surface, or endothelialization, may increase the biocompatibility of an implanted device. For example, a stent which permits the formation of the endothelium on the inside diameter (blood contacting surface) of the stent may further promote healing at the therapeutic region and/or have longer term viability. For example, a stent coated with endothelial cells may be more consistent with the surrounding body lumens, thereby resulting in less turbulent blood flow or a decreased risk of thrombosis, or the formation of blood clots. A stent which permits the formation of an endothelial layer on the inside surface of the stent may therefore be particularly biocompatible, resulting in less trauma at the point of application, fewer side effects, and/or longer term device viability.

Medical appliances including a covering of porous or semi-porous material may be configured to inhibit or reduce inflammatory responses by the body toward the tissue contacting side of the medical appliance, for example. Mechanisms such as an inflammatory response by the body toward the medical appliance may stimulate, aggravate, or encourage negative outcomes, such as neointimal hyperplasia. For example, a device configured to permit tissue ingrowth and/or the growth or attachment of endothelial cells onto the blood contacting side of the device may reduce the likelihood of negative flow characteristics and blood clotting. Similarly, a device so configured may mitigate the body's inflammatory response toward the material on, for example, the tissue or non-blood contacting side of the device. By modulating the evoked inflammatory response, negative outcomes such as the presence of bioactive inflammatory macrophages and foreign body giant cells may be reduced. This may aid in minimizing the chemical chain of responses that may encourage fibrous capsule formation surrounding the device and events stimulating neointimal hyperplasia.

Additionally, medical appliances including a covering of porous or semi-porous material of reduced or diminished wall thickness may be provide certain mechanical characteristics to the medical appliance. For instance, stent grafts including thinner walls of the covering or graft can include improved flexibility, facilitating navigation through vascular anatomies. Furthermore, a reduced wall thickness of a stent graft enhances deliverability by facilitating a smaller profile when crimped, or compressed onto a delivery system. A lower profile, when crimped, minimizes the risk of damaging vessel walls during delivery and deployment, as well as reduces the risk of obstructing blood flow during delivery.

Rotational spun materials, such as those described herein, may be used to comprise portions of medical appliances, such as stents, patches, grafts, and so forth. The present disclosure is applicable to any implantable medical appliance, notwithstanding any specific examples included below. In other words, though particular medical appliances, such as stents or patches, may be referenced in the disclosure and examples below, the disclosure is also analogously applicable to other medical appliances, such as those which comprise a covering or layer of polymeric material.

In some embodiments, rotational spun nanofibers (and/or microfibers) may be configured to permit interaction with nano-scale (and/or micro-scale) body structures, such as endothelial cells. Rotational spinning refers generally to processes involving the expulsion of flowable material from one or more orifices, the material forming fibers which are subsequently deposited on a collector. Examples of flowable materials include dispersions, solutions, suspensions, liquids, molten or semi-molten material, and other fluid or semi-fluid materials. In some embodiments, the rotational spinning processes are completed in the absence of an electric field. For example, one embodiment of a rotational spinning process comprises loading a polymer solution or dispersion into a cup or spinneret configured with orifices on the outside circumference of the spinneret. The spinneret is then rotated, causing (through a combination of centrifugal and hydrostatic forces, for example) the flowable material to be expelled from the orifices. The material may then form a “jet” or “stream” extending from the orifice, with drag forces tending to cause the stream of material to elongate into a small diameter fiber. The fibers may then be deposited on a collection apparatus. Exemplary methods and systems for rotational spinning can be found in U.S. Patent Publication No. US2009/0280325, titled “Methods and Apparatuses for Making Superfine Fibers,” which is herein incorporated by reference in its entirety.

Rotational spinning may be configured to create mats, tubes, or other structures comprised of elongate fibers, including nanofibers (i.e. fibers which are smaller than one micron in diameter) or microfibers (i.e. fibers which are between one micron and one millimeter in diameter). In some instances the fibers may be randomly disposed, while in other embodiments the alignment or orientation of the fibers may be somewhat controlled or follow a general trend or pattern. Regardless of any pattern or degree of fiber alignment, as the fibers are deposited on a collector or on previously deposited fibers; the fibers are not woven, but rather serially deposited on the collector or other fibers. Because rotational spinning may be configured to create a variety of structures, as used herein, the terms “mat” or “non-woven mat or material” is intended to be broadly construed as referring to any such rotational spun structure, including tubes, spheres, and so on.

Accordingly, the present disclosure relates to medical appliances which may have, in certain embodiments, metal scaffolding covered with at least one layer of rotational spun material, such as rotational spun polytetrafluoroethylene (PTFE). Additionally, the present disclosure relates to medical appliances formed of rotational spun materials which may not have scaffolding structures or have scaffolding structures which are not made of metal. More specifically, the present disclosure relates to rotational spun materials of diminished thickness and/or enhanced uniformity of thickness throughout the material. This can facilitate medical appliances, such as covered stent grafts, with an overall reduced wall thickness. It will be appreciated that, though particular structures and coverings are described below, any feature of the scaffolding or covering described below may be combined with any other disclosed feature without departing from the scope of the current disclosure.

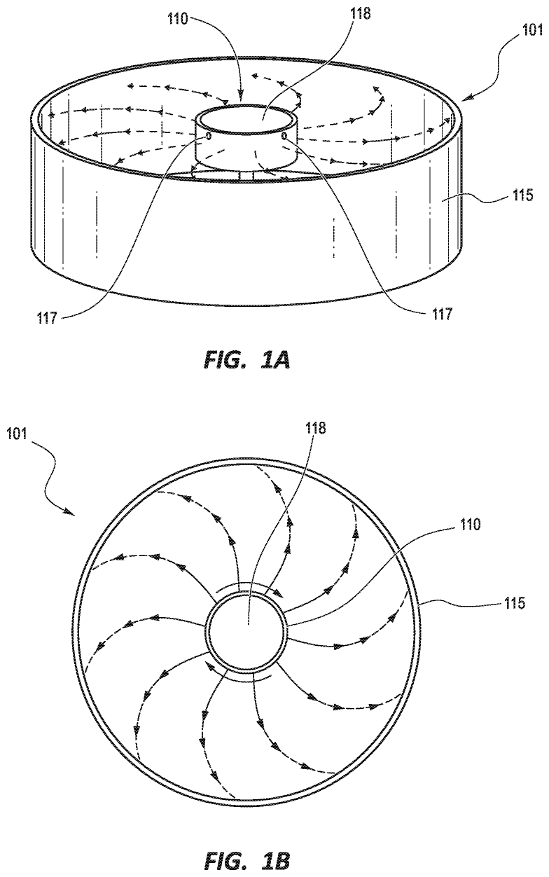

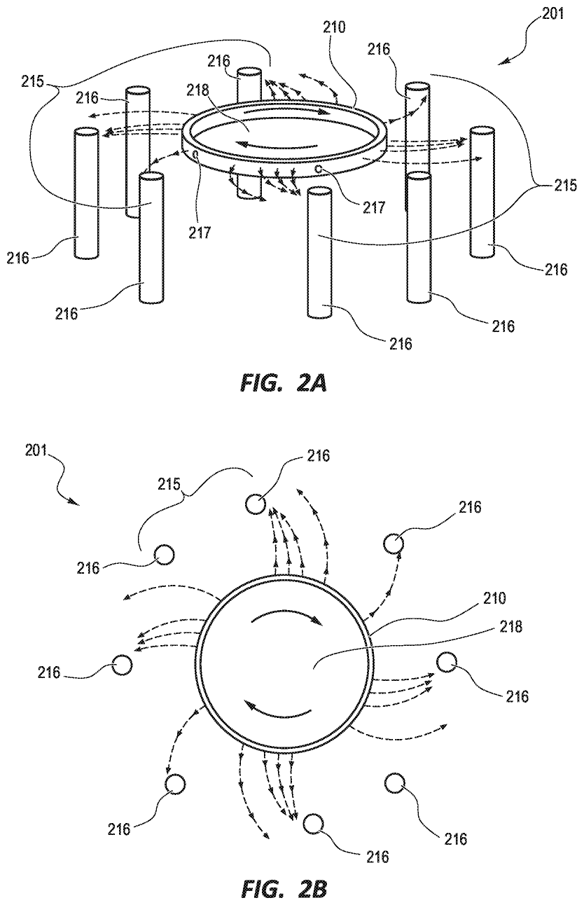

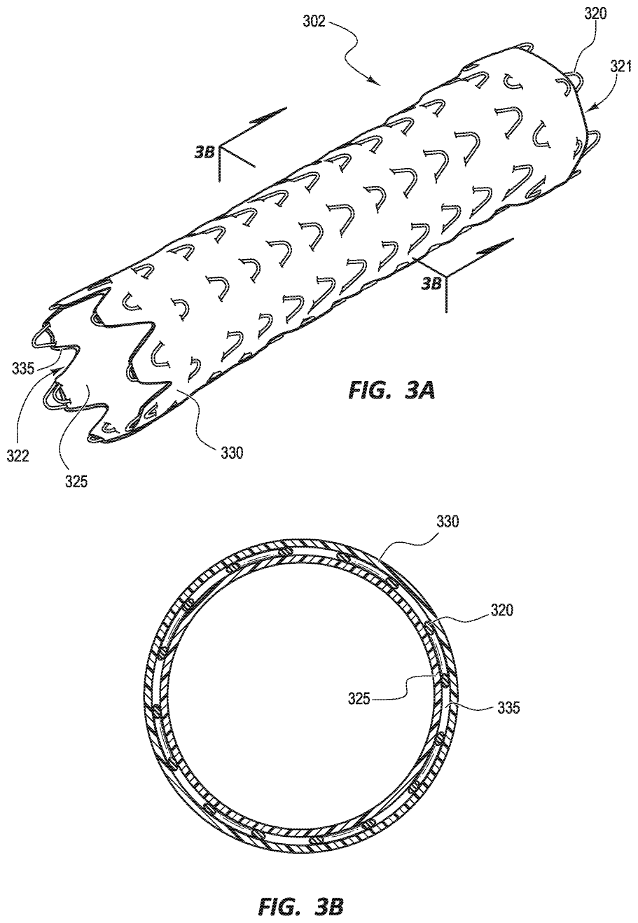

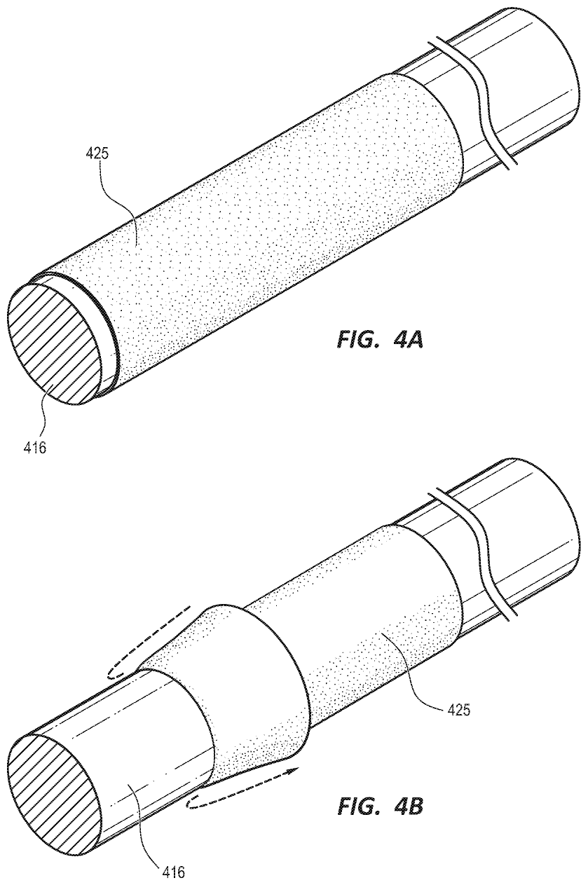

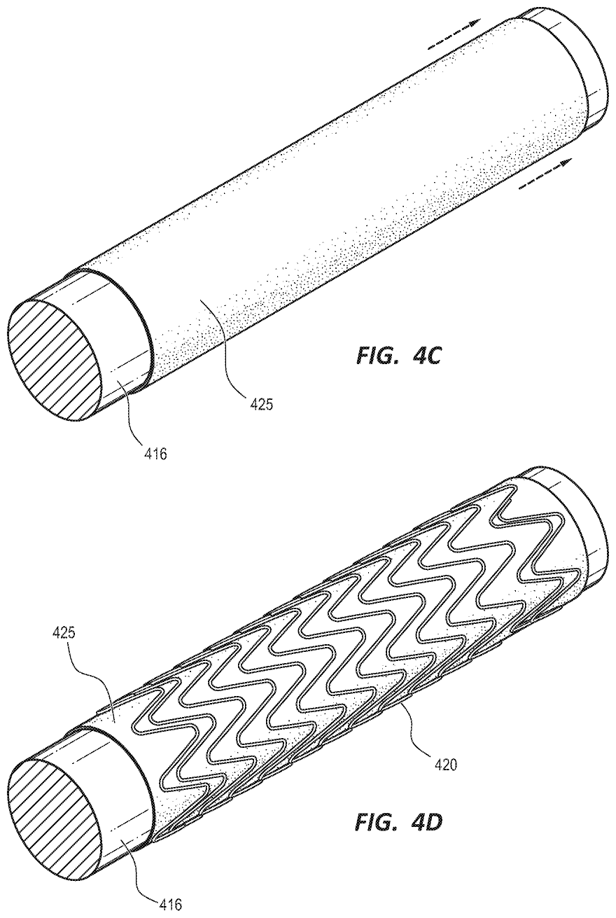

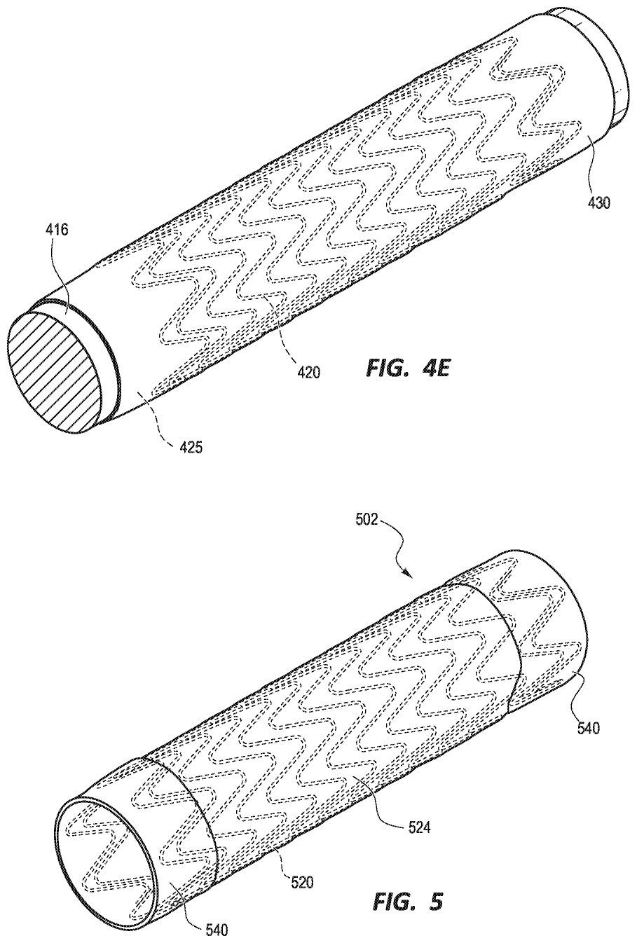

FIGS. 1A, 1B, 2A, and 2B schematically illustrate certain embodiments of rotational spinning apparatuses. FIGS. 3A and 3B illustrate an embodiment of a covered medical appliance. FIGS. 4A-E illustrate certain steps in a process of manufacturing a multi-layered construct of rotational spun materials. FIG. 5 illustrates an embodiment of a medical appliance which includes cuffs at each end of a stent. Again, regardless of whether a medical appliance illustrated in any particular figure is illustrated with a particular covering or coating, or without any covering or coating at all, any embodiment of a medical appliance may be configured with any of the combinations of coverings or coatings shown or described herein.

FIG. 1A illustrates a rotational spinning apparatus 101. This figure, as well as FIGS. 1B, 2A, and 2B, discussed below, are intended to schematically illustrate the operation of a rotational spinning apparatus, and not meant to limit the particular structure, shape, or arrangement of rotational spinning apparatus components within the scope of this disclosure. The illustrated apparatus 101 comprises a spinneret 110 disposed near the center of a generally circular collector 115. In the illustrated embodiment, the collector 115 forms a ring around the spinneret 110. The spinneret 110 further comprises orifices 117 located around the circumference of the spinneret 110 and a reservoir 118.

The apparatus 101 may be utilized to create a mat of rotational spun fibers deposited on the collector 115. In some embodiments, the collector 115 may be configured such that structures such as rods, tubes, or spheres of rotational spun fibers are created.

In some embodiments, apparatus 101 may be utilized to create a mat of rotational spun fibers by first filling the reservoir 118 with a flowable material. In some instances polymer dispersions, including aqueous dispersions or polymer solutions may be used. The spinneret 110 may then be rotated such that the dispersion, or other flowable material, is forced out of the orifices 117 as illustrated by the arrows in FIG. 1A. Molecules, including polymer chains, may tend to disentangle and/or align as the material is forced through the orifice. Additionally, in some embodiments, the orifice 117 comprises a needle or nozzle that extends from the outside circumference of the spinneret 110. Still further, in some embodiments, the orifice 117 may comprise a cannula configured with a quick connection, such as a luer connection, allowing for rapid exchange of various cannula sizes.

As the dispersion is expelled from the reservoir 118, drag or other aerodynamic forces acting on the stream or jet of material may cause the stream of dispersion to elongate and bend, forming a relatively small diameter fiber of material. In some instances drag may be a shear force with respect to the stream. Additionally, certain components of the dispersion, such as the dispersion medium or solvent, may partially or fully evaporate as the material is drawn into fibers. In embodiments utilizing flowable materials which have no solvent, such as molten material, there may be no evaporation as the material is drawn into fibers.

The fibers eventually contact, and are deposited on, collector 115. The combination of forces described above may interact as the fibers are deposited, causing the fibers to be disposed in random patterns on the collector 115. In some embodiments, air currents may be introduced (for example through the use of fans) to partially control the deposition of the fibers on the collector 115.

In embodiments utilizing certain flowable materials, the fibers may then be removed from the collector 115 and sintered, or sintered then removed. For example, sintering may be applicable to PTFE fibers, including PTFE fibers spun from a dispersion. The sintering process may set or bond the structure of the mat and remove any remaining water or other dispersion medium or solvent.

In some embodiments, the mat may be treated at a first temperature to remove solvents and a second temperature to sinter the mat. For example, a PTFE mat spun from an aqueous dispersion may be first treated at a temperature below the sintering temperature of PTFE in order to remove any remaining water. For example, the mat may be heated to about 200 degrees C to remove any remaining water in the mat. Further, other materials such as solvents or fiberizing agents may be evaporated or otherwise driven off at this stage. In some embodiments—as further detailed below—a PTFE dispersion may be mixed with polyethylene oxide (PEO) prior to rotational spinning the mat. As also discussed in the examples below, concentrations of PEO to 60 wt % PTFE dispersion from about 0.04 g/ml to about 0.12 g/ml, including from about 0.06 g/ml to about 0.08 g/ml may be used in some embodiments. In some instances, very high or very low concentrations of PEO may lead to shrinkage during sintering or sputtering during rotational spinning of the material.

Treating the spun mat at temperatures such as 200 degrees C may force off remaining PEO as well as water. In some embodiments the PTFE mat may then be sintered at about 385 degrees C. In other embodiments, PTFE sintering may be completed at temperatures from about 360 degrees C to about 400 degrees C, and/or at temperatures in excess of the crystalline melt point of the PTFE (about 342 degrees C). In other instances the mat may only be heated to the sintering temperature, removing the remaining water and/or PEO while simultaneously sintering the PTFE. Additionally or alternatively, in some embodiments solvents or other materials may be removed by rinsing the mat.

Sintering may set the structure of the mat even if the temperature at which the material is sintered is not sufficient to cause cross linking of the polymer chains. PTFE sintering may create solid, void free, PTFE fibers.

FIG. 1B is a top view of the rotational spinning apparatus 101 of FIG. 1A, illustrating the spinneret 110, the collector 115, and the reservoir 118. In the illustration of FIG. 1B potential arced paths of the streams of material interacting with drag forces are illustrated by arrows and dotted lines. These lines are exemplary and not intended to show the precise path of the fibers. In many embodiments, the fibers may loop completely around the spinneret 110 before contacting the collector 115, including embodiments where the fiber path encircles the spinneret 110 more than one time before contacting the collector 115.

The distance between the spinneret 110 and the collector 115 may impact the diameter of the fibers. In some embodiments, the longer the fibers are drawn out before contacting the collector 115, the smaller the resulting fiber diameters. Similarly, smaller distances may be configured to produce larger diameter fibers.

Processes such as the exemplary process described above may be utilized to create structures comprised of small diameter fibers, including nanofibers. The fiber mat may then be incorporated into a medical appliance configured for implantation in the human body. Some such structures, including nanofiber structures, may be configured to permit tissue ingrowth and/or endothelial growth or attachment on the mat. For example the mat may be configured with openings within the fibers or similar structures configured to permit interaction with tissue and/or cells. As further detailed below, the percent porosity of a fiber mat, the thickness of the mat, and the diameter of the fibers comprising the mat may each be configured to create a fiber mat with desired properties, including mats that tend to permit or resist tissue ingrowth and/or endothelial growth or attachment.

A number of variables may be controlled to affect the properties of a rotational spun mat. Some of these variables include: the rotational speed of the spinneret; the viscosity of the solution, dispersion, or other flowable material; the temperature of the spinneret; introduced air currents; the thickness of the mat; and so on. In the case of fibers spun from molten material, the melt flow index (MFI) of the material may also impact the nature of the spun mat. In some embodiments, materials with an MFI of from about 1 g/10 min to about 5000 g/10 min, including from about 200 g/10 min to about 1500 g/10 min and from about 10 g/10 min to about 30 g/10 min, will tend to form fibers when spun.

In other embodiments a rotational spun mat may be configured to resist tissue ingrowth into or through the mat. In such embodiments, the mat may be configured with very small pores, or essentially no pores at all, thus preventing tissue ingrowth into or through the mat. Certain medical appliances may be constructed partially of rotational spun materials configured to permit tissue ingrowth and/or endothelial growth or attachment and partially of rotational spun materials configured to resist tissue ingrowth and/or attachment. Characteristics of the rotational spun fiber mat, such as porosity and average pore size, may be controlled during the rotational spinning process to create certain mats which permit tissue ingrowth and/or endothelial growth or attachment and other mats which resist or are impermeable to tissue ingrowth and/or attachment.

In some embodiments, a PTFE dispersion may be used to rotational spin a mat or another structure comprised of PTFE nanofibers. Furthermore, in some exemplary embodiments PEO may be added to the PTFE dispersion prior to rotational spinning the material. The PEO may be added as a fiberizing agent, to aid in the formation of PTFE fibers within the dispersion or during the process of rotational spinning the material. In some instances the PEO may more readily dissolve in the PTFE dispersion if the PEO is first mixed with water. In some examples this increased solubility may reduce the time needed to dissolve PEO in a PTFE dispersion from as long as multiple days to as little as 30 minutes. After material is rotational spun onto a collector, the material may then be sintered as further described below. In some instances the sintering process can tend to set or harden the structure of the PTFE. Furthermore, as described above, sintering may also eliminate the water and PEO, resulting in a mat of substantially pure PTFE. Additionally, as also described above, the mat may first be heat treated at a temperature below the sintering temperature of the PTFE, in order to remove water and/or PEO from the mat. In some embodiments this step may be completed at about 200 degrees C.

The water, PEO, and PTFE amounts may be controlled to optimize the viscosity, PEO/PTFE ratio, or other properties of the mixture. In some instances adding water to the PEO before mixing with the PTFE dispersion may aid in reducing the number of solid chunks in the mixture, lower the preparation time for the mixtures, and reduce the time needed for the combined mixture to solubilize.

A variety of materials may be rotational spun to form structures for use in medical appliances. Exemplary materials which may be rotational spun for use in implantable appliances include PTFE, fluorinated ethylene propylene (FEP), Dacron or Polyethylene terephthalate (PET), polyurethanes, polycarbonate polyurethanes, polypropylene, Pebax, polyethylene, biological polymers (such as collagen, fibrin, and elastin), and ceramics.

Furthermore, additives or active agents may be integrated with the rotational spun materials, including instances where the additives are directly rotational spun with other materials. Such additives may include radiopaque materials such as bismuth oxide, antimicrobial agents such as silver sulfadiazine, antiseptics such as chlorhexidine or silver and anticoagulants such as heparin. Organic additives or components may include fibrin and/or collagen. In some embodiments, a layer of drugs or other additives may be added to a rotational spun appliance during manufacture. Additionally, some appliances may be constructed with a combination of synthetic components, organic components, and/or active ingredients including drugs, including embodiments wherein an appliance is comprised of alternating layers of these materials. Moreover, in some embodiments a medical appliance may consist of layers of rotational spun materials configured to control the release of a drug or another active layer disposed between such layers. Active layers or ingredients such as drugs or other active agents may be configured to reduce or otherwise modify or influence the biological response of the body to the implantation of the medical appliance.

Additionally, in some embodiments the material supplied to the reservoir 118 may be continuously supplied (for example by a feed line), including embodiments where the reservoir is pressurized or supplied by a pressurized source. Furthermore, in some embodiments the material may be heated near or above its melting point prior to rotational spinning, including embodiments wherein the material is melted and not dispersed in a solvent. Thus, in some embodiments, rotational spinning molten material does not include the use of solvents; therefore there is no need to remove solvents from the mat at a later step in the process. In some instances the material may be supplied to the reservoir as pellets which are heated and melted within the reservoir.

Still further, in some instances collector 115 may have an electrostatic charge. Additionally, in some embodiments rotational spun structures may be combined with electrospun structures, including embodiments where some layers of material are rotational spun and some electrospun, but both deposited on the same substrate or construct. Electrospinning, and its use in connection with medical appliances, is described in U.S. patent application Ser. No. 13/360,444, filed on Jan. 27, 2012 and titled “Electrospun PTFE Coated Stent and Method of Use,” which is hereby incorporated by reference in its entirety.

Referring specifically to FIGS. 2A-B, another schematic embodiment of a rotational spinning apparatus 201 is illustrated. FIGS. 2A-B illustrate an apparatus analogous to that shown in FIGS. 1A-B. It will be appreciated by one of skill in the art having the benefit of this disclosure that analogous components of the two apparatuses may be interchangeable and that disclosure provided in connection with each embodiment may be applicable to the other and vice versa.

FIG. 2A is a perspective view of the rotational spinning apparatus 201 while FIG. 2B is a top view of the same. Rotational spinning apparatus 201 includes a spinneret 210 comprising a reservoir 218 and orifices 217. As compared to the apparatus 101 of FIGS. 1A-B, in the embodiment of FIGS. 2A-B, collector 115 is configured as a plurality of cylindrical mandrels 216. Thus in FIGS. 2A-B the plurality of mandrels 216 are collectively designated as a collector 215, but individually designated by the numeral 216. The term “collector” as used in connection with FIGS. 1A-2B, and indicated by numerals 115 and 215, is intended to broadly refer to any collection device or apparatus without defining a particular size, shape, or orientation. For example, in some embodiments the collector may be configured as a ring, such as collector 115 illustrated in FIGS. 1A-B. In other embodiments collector 215 may be a plurality of cylinders as shown in FIGS. 2A-B. In still other embodiments, the collector may comprise a rotating belt (not shown), configured to facilitate rotational spinning of a continuous sheet of material.

Embodiments configured to form a continuous sheet of rotational spun material may be configured to produce mats, including mats from about one meter to about 9 meters in width, such as mats of about 3 meters in width. Mats from about one foot wide to about one meter wide (as well as larger or smaller mats) may be formed. In some instances, a sintering oven may be positioned such that as the mat moves away from the spinneret (on the belt) the mat enters the oven and is sintered. The sintered mat may then be collected onto a spool. Further, in some embodiments, the entire spool may then be cut into smaller widths, forming strips of material. For example, strips from about 0.1 inch wide to about 2 inches wide may be formed. Such strips may be utilized for the construction of tubular appliances by wrapping the strips around a mandrel. The strips may overlap and/or may be wound such that the tube formed does not have a distinct seam along the length of the tube. In some instances, the mat may be wound in multiple layers around the mandrel. Further, the mat formed may be relatively thin, or film-like. The thickness of the covering formed on the mandrel (and other characteristics such as porosity) may be controlled by the number of layers of film wound onto the mandrel.

In some embodiments, rotational spun tubular medical devices, such as stents, may comprise one or multiple bifurcations or branches. Thus, medical devices which comprise a single lumen which splits or bifurcates into two or more lumens are within the scope of this disclosure. Likewise, medical appliances comprising a main lumen with one or multiple branch lumens extending from the wall of the main lumen are within the scope of this disclosure. For example, a thoracic stent-configured for deployment within the aorta-may comprise a main lumen configured to be disposed in the aorta and branch lumens configured to extend into side branch vessels originating at the aorta. Similarly, in some embodiments such stents may alternately be configured with access holes in the main lumen configured to allow access (possibly for additional stent placement) and flow from the main vessel to any branch vessels extending there from.

In some embodiments, a bifurcated medical appliance may be manufactured by first creating a bifurcated mandrel in which the bifurcated mandrel portions are removable from the portion of the mandrel coinciding with the main lumen. The leg or branch portions of the mandrel may be splayed 180 degrees apart with a common axis of rotation. Thus, in some embodiments, the entire mandrel may form a T-shape. The entire mandrel may then be rotated about the axis of the leg portions and rotational spun fibers collected on the leg portions of the mandrel. The mandrel may then be oriented to rotate about the axis of the main lumen portion of the mandrel, and any unwanted fibers disposed while spinning on the bifurcated leg portions may be wiped off. The mandrel may then be rotated about the axis of the main lumen portion and fibers collected on the main lumen portion of the mandrel. The entire mandrel may then be placed in an oven and sintered. The mandrel portions associated with the bifurcated legs may then be removed from the leg or branch portions of the appliance, and the single lumen mandrel portion subsequently removed from the spun appliance. The appliance may then be placed on or within a frame structure, such as a stent frame. A dip or film coating (such as of FEP or PTFE) may then be applied over the construct to create an impervious outside layer and/or to further bond the frame to the spun portion of the appliance.

In any of the exemplary embodiments or methods disclosed herein, in instances where the nanofibers are formed of PTFE, the sintering temperature may be from about 360 degrees C to about 400 degrees C, including at temperatures of about 385 degrees C or at temperatures above the crystalline melting temperature of the PTFE, or about 342 degrees C. Similarly, for other materials, sintering may be done at or above the crystalline melting temperature of other spun polymers. Again, either prior to or as part of the sintering process, heat treating may be configured to remove PEO and/or water, in instances where the PTFE or other polymer was combined with such elements prior to spinning the mat.

In the embodiment of FIGS. 2A-B, mandrels 216 may be disposed about spinneret 210 in a generally circular configuration. In some embodiments, mandrels 216 may be stationary while in other embodiments the mandrels 216 may be configured to rotate about their axes. In some such embodiments the mandrels 216 may each be driven by the same belt, allowing each to maintain the same rotational speed. In other embodiments some or all of the mandrels 216 may be independently driven.

In the illustrated embodiment, mandrels 216 are disposed vertically, or such that the axis of each mandrel is substantially parallel to the axis of rotation of the spinneret. In another exemplary embodiment, one or more of the mandrels 216 may be disposed horizontally, or such that the axes of those mandrels is substantially orthogonal to the axis of rotation of the spinneret. In some embodiments, the axes of one or more of mandrels 216 may be generally parallel to the axes of fibers being spun. Horizontally disposed mandrels 216 may be configured to produce mats having generally less fiber alignment than vertical mandrels. Horizontal mandrels may further be configured to produce mats with relatively uniform thickness around the mandrel.

In addition to horizontal mandrels, further embodiments may comprise mandrels disposed in any relative position with respect to the axis of the spinneret. Mandrels mounted in any disposition may be configured as stationary collection devices or configured to rotate. Additionally, combinations of mandrels in a variety of positions may be used simultaneously. Furthermore, in some embodiments one or more mandrels 216 may be configured for use in connection with a vacuum system. For example, openings in the surface of the mandrel, such a micro-porous mandrels 216, may tend to draw fibers toward the mandrel in instances where the interior of the mandrels has lower pressure than the exterior of the mandrels.

In embodiments wherein mandrels 216 rotate, the spinning motion of each mandrel 216 may tend to deposit the fibers around the entire surface of the mandrel. Thus, as the fibers are deposited on each mandrel 216, a seamless tube of nanofiber material may form on each mandrel 216. The density of the fibers, the thickness of the mat, and other characteristics may be controlled by such variables as the distance from the spinneret 210 to the mandrels 216, the rotational speed of the spinneret 210, the rotational speed of the mandrels 216, the orientation of the mandrels 216, the characteristics of the solution being spun, and so forth. In some instances, mats of rotational spun material formed on a spinning mandrel 216 may thus comprise a tubular membrane having no seam and substantially isotropic properties. In some instances, the collection mandrel 216 may rotate at rates between about 1 RPM and about 2000 RPM during the rotational spinning process, including rates from about 1000 RPM to about 1500 RPM, including about 1500 RPM, or about 50 RPM to about 300 RPM, including about 150 RPM. In some instances, the rotational speed of one or more collection mandrels may be related to the rate at which the apparatus produces fibers. For example, in some embodiments, faster mandrel rotational speed may be correlated with higher total fiber production rates for the apparatus.

Furthermore, controlling the rotational speed of the mandrels 216 may influence both the density of the mat formed on the mandrels 216 and the general alignment of fibers in the mat. For instance, in some embodiments utilizing vertical mandrels, the faster the mandrel 216 is spinning the more the fibers may tend to be deposited in-line with other fibers. Further, the relative density of the fibers, for example, as measured by percent porosity, may be controlled in part by the rotational speed of the mandrels 216.

As further detailed in connection with FIGS. 4A-E, once the fibers are rotational spun onto the mandrels 216 the fibers may be sintered. In some embodiments a scaffolding structure, such as a stent wire, may also be on the mandrels 216, and the nanofibers rotational spun directly onto the mandrels 216 and scaffolding structure.

FIGS. 3A-B illustrate an exemplary medical appliance: stent 302. Stent 302 comprises a scaffolding structure 320 and a covering comprising an inner layer 325, an outer layer 330, and a tie layer 335. In other embodiments, a stent covering may have more or fewer layers than the illustrated embodiment, including embodiments with only one covering layer. Again, disclosure recited herein with respect to specific medical appliances, such as stents, may also be applicable to other medical appliances.

The cover of the stent 302 of FIG. 3A comprises a flat end 321 and a scalloped end 322. At the flat end 321 of the illustrated embodiment, the cover of the stent 302 is cut substantially perpendicular to the longitudinal axis of the stent 302. At the scalloped end 322, the cover of the stent 302 comprises cut away, or scalloped, portions at the end of the stent 302. Scalloped ends may be configured to reduce infolding of the stent cover at the ends. For example, in some instances, a stent may have a larger diameter than a vessel in which it is deployed. Thus, the vessel may partially compress the stent radially. In some instances this radial compression may create folds or wrinkles in flat cut stent covers. These folds may then impede blood flow or lead to clotting within the vessel. Scalloped ends may reduce the occurrence of infolding at the end of a radially compressed stent. It is within the scope of this disclosure to use either type of end on any end of any stent.

Membranes composed of rotational spun mats may have a microstructure composed of many fibers crossing each other at various and random points. The rotational spinning process may control the thickness of this structure and thereby, the relative permeability of the mat. As more and more fibers are rotational spun onto a mat, the mat may both increase in thickness and decrease in permeability (due to successive layers of strands occluding the pores and openings of layers below).

Mats produced in connection with the present disclosure may be described by three general parameters: percent porosity, mat thickness, and fiber diameter. Each of these parameters may impact the nature of the mat, including the tendency of the mat to permit tissue ingrowth and/or endothelial attachment or the tendency of the mat to resist tissue ingrowth or endothelial attachment. Each of these parameters may be optimized with respect to each other to create a mat having particular characteristics.

Percent porosity refers to the percent of open space to closed space (or space filled by fibers) in a fiber mat. Thus, the more open the mat is, the higher the percent porosity measurement. In some instances, percent porosity may be determined by first obtaining an image, such as an SEM, of a rotational spun material. The image may then be converted to a “binary image,” or an image showing only black and white portions, for example. The binary image may then be analyzed and the percent porosity determined by comparing the relative numbers of each type of binary pixel. For example, an image may be converted to a black and white image wherein black portions represent gaps or holes in the rotational spun mat while white portions represent the fibers of the mat. Percent porosity may then be determined by dividing the number of black pixels by the number of total pixels in the image. In some instances, a code or script may be configured to make these analyses and calculations.

In some embodiments, percent porosities from about 30% to about 80% may be configured to permit tissue ingrowth into the layer and/or permit endothelial growth or attachment on the layer, including mats of about 40% to about 60%, mats of about 45% to about 50%, or mats of about 50% porosity. Less open layers may be configured to resist such ingrowth and/or attachment. Because the fibers comprising the mat are deposited in successive layers, the second parameter, mat thickness, may be related to porosity. In other words, the thicker the mat, the more layers of fibers, and the less porous the mat may be. In some embodiments, mats from about 20 micrometers to about 100 micrometers may be configured for use in connection with the present disclosure, including mats from about 40 micrometers to about 80 micrometers. Finally, the third parameter, fiber diameter, may be a measurement of the average fiber diameter of a sample in some instances. In some embodiments fiber diameters from about 50 nanometers to about 3 micrometers may be used in connection with the present disclosure. Notwithstanding these or other specific ranges included herein, it is within the scope of this disclosure to configure a mat with any combination of values for the given parameters.

In some embodiments the “average pore size” of the mat may be used as an alternate or additional measurement of the properties of the mat. The complex and random microstructure of rotational spun mats presents a challenge to the direct measurement of the average pore size of the mat. Average pore size can be indirectly determined by measuring the permeability of the mat to fluids using known testing techniques and instruments. Once the permeability is determined, that measurement may be used to determine an “effective” pore size of the rotational spun mat. As used herein, the “pore size” of a rotational spun mat refers to the pore size of a membrane which corresponds to the permeability of the rotational spun mat when measured using ASTM standard F316 for the permeability measurement. This standard is described in ASTM publication F316 “Standard Test Methods for Pore Size Characteristics of Membrane Filters by Bubble Point and Mean Flow Pore Test,” which is incorporated herein by reference. In some instances this test can be used as a quality control after configuring a mat based on the three parameters (percent porosity, thickness, and fiber diameter) discussed above.

In some applications it may be desirable to create a medical appliance such as stent 302 with an outer layer 330 which is substantially impermeable. Such a layer may decrease the incidence of lumen tissue surrounding the stent growing into or attaching to the stent. This may be desirable in applications where the stent is used to treat stenosis or other occlusions; an impermeable outer layer may prevent tissue from growing into or through the material toward or into the lumen of the stent and reblocking or restricting the body lumen.

In some embodiments a substantially impermeable outer layer may be produced by using rotational spun mats with a percent porosity from about 0% to about 50%, including about 25%, and fiber diameters from about 50 nanometers to about 3 micrometers.

In some embodiments a substantially impermeable outer layer may be produced by using rotational spun mats. Some mats produced by rotational spinning may have parameters, such as thickness, which vary along one or more dimension of the mat. For instance, this method can involve creating mats where the thickness varies along a first dimension of the mat, such as the length or width. Variability in thickness throughout the mat can be controlled during the spinning process, allowing for management of the mat's uniformity or isotropic characteristics.

In some embodiments a substantially impermeable outer layer may be produced by using rotational spun mats with a thickness from about 20 micrometers to about 120 micrometers, from about 30 micrometers to about 110 micrometers, or from about 40 micrometers to about 100 micrometers. More specifically, in some cases, an average thickness of the mat can be anywhere within the range from about 20 micrometers to about 120 micrometers, from about 30 micrometers to about 110 micrometers, or from about 40 micrometers to about 100 micrometers. In some cases, a substantially impermeable outer layer may be produced by using rotational spun mats with a thickness from about 30 micrometers to about 300 micrometers. In some cases, a substantially impermeable outer layer may be produced by using rotational spun mats with a thickness from about 30 micrometers to about 200 micrometers.

In embodiments, the thickness of the mat can vary no more than 100 micrometers, or 80 micrometers, or 60 micrometers, or 40 micrometers, or 20 micrometers, along a first dimension of the mat (e.g., length or width). Otherwise stated, in embodiments, a maximum thickness of the mat can be no more than 100 micrometers, 80 micrometers, or 60 micrometers, or 40 micrometers, or 20 micrometers, more than a minimum thickness of the mat.

In embodiments, a maximum thickness of the mat can be no more than 180%, 160%, or 140% of an average thickness of the mat. A minimum thickness of the mat can be no less than 60%, 40%, or 20% of an average thickness of the mat.

In some embodiments, a standard deviation of the mat thickness can be no more than 60 micrometers, 40 micrometers, or 20 micrometers, or 15 micrometers, or 10 micrometers.

Additionally, or alternatively, a substantially impermeable mat may have an average pore size of about 0 micrometers to about 1.5 micrometers. In other embodiments, the impermeable layer may have an average pore size of less than about 0.5 micron. In yet other embodiments, the impermeable layer may have an average pore size of less than about 1 micron. In some embodiments, the impermeable layer may be a layer other than the outer layer, such as a tie layer, an intermediate layer, or an inner layer.

In one example, a medical appliance such as stent 302 may be covered with a rotational spun PTFE inner layer 325 and a rotational spun PTFE outer layer 330. The outer layer 330 may be configured to be substantially impermeable to tissue ingrowth and/or attachment. In other embodiments the impermeability of the stent may be provided by a tie layer 335 disposed between the outer layer 330 and the inner layer 325. For example, a substantially impermeable layer may be formed of FEP which is applied, for example, as a film or dip coating between rotational spun layers of PTFE. Furthermore, FEP may be rotational spun with a small average pore size to create a substantially impermeable layer. In some embodiments both the outer layer 330 and the tie layer 335 may be configured to be substantially impermeable.

Dip coatings may be applied by dipping a portion of a layer or construct in a polymer dispersion. For example, a PTFE layer may be dip coated on a construct by adding 20 ml of water to 50 ml of a 60 wt % PTFE dispersion to thin the dispersion. A fiber mat may then dipped in the solution to coat the mat. The dip coat may then sintered at 385 degrees C for 15 minutes. Other concentrations of PTFE dispersions for dip coatings are also within the scope of this disclosure.

Further, an FEP layer may be dip coated on a construct by adding 20 ml of water to 50 ml of a 55 wt % dispersion to thin the dispersion. A fiber mat may then dipped in the solution to coat the mat. The dip coat may then cooked, for example, at 325 degrees C for 15 minutes. Other concentrations of FEP dispersions for dip coatings are also within the scope of this disclosure. Additionally, polymer dispersions may be sprayed or otherwise applied onto a surface (such as a fiber mat) to coat the surface. Such coatings may be heat treated after application.

In some embodiments, more or less water, for example from about 10 ml to about 50 ml, may be added to similar amounts and concentrations of the dip dispersions above to thin the dispersions. Additionally, substances other than, or in addition to, water may be used to thin a dispersion for dip coating. For example, a surfactant or a solvent may be used. In some such cases the surfactant or solvent may later be removed from the construct, including embodiments where it is allowed to evaporate when the coat is sintered or cooked. Alcohols, glycols, ethers, and so forth may be so utilized.

In some embodiments it may be desirable to create a medical appliance such as stent 302 with an outer layer 330 which is more porous. A porous outer layer 330 may permit healing and the integration of the prosthesis into the body. For instance, tissue of the surrounding lumen may grow into the porous outer diameter or attach to the outer diameter layer. This tissue ingrowth may permit, modulate, and/or influence healing at the therapy site. In some embodiments a porous outer layer 330 may be formed of rotational spun PTFE.

In certain embodiments a relatively porous inner layer 325 may be desirable. This layer may or may not be used in conjunction with a substantially impermeable outer layer 330. A relatively porous inner layer may permit tissue ingrowth and/or endothelial attachment or growth on the inside diameter of the stent 302 which may be desirable for any combination of the following: healing, biocompatibility, prevention of thrombosis, and/or reducing turbulent blood flow within the stent. In some embodiments the inner layer may be comprised of a mat, such as a rotational spun PTFE mat, having a percent porosity of about 40% to about 80%, including about 50%; a thickness of about 20 micrometers to about 100 micrometers, including from about 40 micrometers to about 80 micrometers; and fiber diameters from about 50 nanometers to about 3 micrometers.

In some embodiments a relatively porous inner layer and/or outer layer may be produced by using rotational spun mats. Some mats produced by rotational spinning may have parameters, such as thickness, which vary along one or more dimension of the mat. For instance, this method can involve creating mats where the thickness varies along a first dimension of the mat, such as the length or width. Variability in thickness throughout the mat can be controlled during the spinning process, allowing for management of the mat's uniformity or isotropic characteristics.

In some embodiments a relatively porous inner layer and/or outer layer may be produced by using rotational spun mats with a thickness from about 20 micrometers to about 120 micrometers, from about 30 micrometers to about 110 micrometers, or from about 40 micrometers to about 100 micrometers. More specifically, in some cases, an average thickness of the mat can be anywhere within the range from about 20 micrometers to about 120 micrometers, from about 30 micrometers to about 110 micrometers, or from about 40 micrometers to about 100 micrometers. In some embodiments a relatively porous inner layer and/or outer layer may be produced by using rotational spun mats with a thickness from about 30 micrometers to about 300 micrometers. In some cases, the relatively porous inner layer and/or outer layer may be produced by using rotational spun mats with a thickness from about 30 micrometers to about 200 micrometers.

In embodiments, the thickness of the mat can vary no more than 100 micrometers, or 80 micrometers, or 60 micrometers, or 40 micrometers, or 20 micrometers, along a first dimension of the mat (e.g., length or width). Otherwise stated, in embodiments, a maximum thickness of the mat can be no more than 100 micrometers, 80 micrometers, or 60 micrometers, or 40 micrometers, or 20 micrometers, more than a minimum thickness of the mat.

In embodiments, a maximum thickness of the mat can be no more than 180%, 160%, or 140% of an average thickness of the mat. A minimum thickness of the mat can be no less than 60%, 40%, or 20% of an average thickness of the mat.

In some embodiments, a standard deviation of the mat thickness can be no more than 60 micrometers, 40 micrometers, or 20 micrometers, or 15 micrometers, or 10 micrometers.

Additionally or alternatively the mat may be comprised of a rotational spun mat, such as PTFE, with an average pore size of about 1 micrometers to about 12 micrometers, such as from about 2 micrometers to about 8 micrometers, or from about 3 micrometers to about 5 micrometers, or alternatively from about 3.5 micrometers to about 4.5 micrometers.

FIG. 3B illustrates a cross-sectional view of the stent 302 of FIG. 3A, again comprising a scaffolding structure 320 and covering comprising an inner layer 325, an outer layer 330, and a tie layer 335. Though in the illustration of FIG. 3B the tie layer 335 is shown at the same “level” as the scaffolding structure 320, the tie layer may be above or below the scaffolding in some embodiments. Further, as shown in FIG. 3B, each layer of the covering may be disposed so that there are no voids between layers.

In some embodiments the tie layer 335 may be configured to promote bonding between the outer layer 330 and the inner layer 325. In other embodiments the tie layer 335 may further be configured to provide certain properties to the stent 302 as a whole, such as stiffness or tensile strength. The tie layer 335 may thus be configured as a reinforcing layer. In some embodiments, expanded PTFE (ePTFE) may be configured as a reinforcing layer. ePTFE may be anisotropic, having differing properties in differing directions. For example, ePTFE may tend to resist creep in the direction the ePTFE membrane was expanded. A reinforcing layer of ePTFE may be oriented to increase strength, resist creep, or impart other properties in a particular direction. ePTFE may be oriented such that the expanded direction is aligned with an axial direction of a medical device, a transverse direction, a radial direction, at any angle to any of these directions, and so forth. Similarly, multiple layers of ePTFE may be disposed to increase strength, resist creep, or impart other properties in multiple directions. The reinforcing layer may or may not be impermeable.

Additionally, in embodiments where both the inner layer 325 and the outer layer 330 are porous in nature, the tie layer 335 may be configured to create an impermeable layer between the two porous layers. In such embodiments the stent may permit tissue ingrowth, tissue attachment and/or healing on both the inner and outer surfaces of the stent while still preventing tissue outside of the stent from growing into the lumen and occluding the lumen. Thus, tie layers may be configured to create a mid-layer portion of a construct, the tie-layer configured to inhibit tissue ingrowth into the layer or to be impervious to tissue migration into or through the layer or to substantially inhibit tissue migration.

Furthermore, the tie layer 335 may be configured to be impervious or substantially impervious to fluid migration across the tie layer 335. Specifically, constructions comprising one or more porous layers may allow fluid to cross the porous layer. In the case of a medical appliance configured to control blood flow, such as a graft, a porous layer may allow blood to leak across the layer or may allow certain smaller components of the blood to cross the layer while containing larger components, effectively filtering the blood. In some instances this filtration or ultrafiltration may allow components such as plasma to cross the barrier while containing red blood cells, leading to seroma. Thus, a fluid impermeable tie layer may be configured to contain fluid within a medical device also comprised of porous layers. In some devices, a tie layer may be both fluid impermeable and impervious to tissue ingrowth, or may be configured with either of these properties independent of the other. Constructs wherein any layer (other than, or in addition to a tie layer) is configured to be fluid impermeable and/or impervious to tissue ingrowth are also within the scope of this disclosure. Thus, disclosure recited herein in connection with fluid impermeable and/or tissue impervious tie layers may be analogously applied to impermeable layers at various locations within a construct.

The tie layer (or any impermeable/impervious layer) may include any thermoplastic and may or may not be rotational spun. In one embodiment, the tie layer may be expanded PTFE. In another it may be rotational spun PTFE. In other embodiments it may be FEP, including rotational spun FEP and FEP applied as a film or dip coating. Furthermore, the tie layer may include any of the following polymers or any other thermoplastic: dextran, alginates, chitosan, guar gum compounds, starch, polyvinylpyridine compounds, cellulosic compounds, cellulose ether, hydrolyzed polyacrylamides, polyacrylates, polycarboxylates, polyvinyl alcohol, polyethylene oxide, polyethylene glycol, polyethylene imine, polyvinylpyrrolidone, polyacrylic acid, poly(methacrylic acid), poly(itaconic acid), poly(2-hydroxyethyl acrylate), poly(2-(dimethylamino)ethyl methacrylate-co-acrylamide), poly(N-isopropylacrylamide), poly(2-acrylamido-2-methyl-l-propanesulfonic acid), poly (methoxyethylene), poly(vinyl alcohol), poly(vinyl alcohol) 12% acetyl, poly(2,4-dimethyl-6-triazinylethylene), poly(3morpholinylethylene), poly(N-l,2,4-triazolyethylene), poly (vinyl sulfoxide), poly(vinyl amine), poly(N-vinyl pyrrolidone-co-vinyl acetate), poly(g-glutamic acid), poly(Npropanoyliminoethylene), poly(4-amino-sulfo-aniline), poly [N-(p-sulphophenyl)amino-3-hy droxymethyl-1,4phenyleneimino-l,4-phenylene], isopropyl cellulose, hydroxyethyl, hydroxylpropyl cellulose, cellulose acetate, cellulose nitrate, alginic ammonium salts, i-carrageenan, N-[(3′-hydroxy-2′,3′-dicarboxy)ethyl]chitosan, konjac glocomannan, pullulan, xanthan gum, poly(allyammonium chloride), poly(allyammonium phosphate), poly(diallydimethylammonium chloride), poly(benzyltrimethylammonium chloride), poly(dimethyldodecyl(2-acrylamidoethyly) ammonium bromide), poly(4-N-butylpyridiniumethylene iodine), poly(2-N-methylpridiniummethylene iodine), poly(N methylpryidinium-2,5-diylethenylene), polyethylene glycol polymers and copolymers, cellulose ethyl ether, cellulose ethyl hydroxyethyl ether, cellulose methyl hydroxyethyl ether, poly(l-glycerol methacrylate), poly(2-ethyl-2-oxazoline), poly(2-hydroxyethyl methacrylate/methacrylic acid) 90:10, poly(2-hydroxypropyl methacrylate), poly(2-methacryloxyethyltrimethylammonium bromide), poly(2-vinyl1-methylpyridinium bromide), poly(2-vinylpyridine N-oxide), poly(2-vinylpyridine), poly(3-chloro-2-hydroxypropyl 2-methacryloxyethyldimethylammonium chloride), poly(4vinylpyridine N-oxide), poly(4-vinylpyridine), poly (acrylamide/2-methacryloxyethyltrimethylammonium bromide) 80:20, poly(acrylamide/acrylic acid), poly(allylamine hydrochloride), poly(butadiene/maleic acid), poly(diallyldimethylammonium chloride), poly(ethyl acrylate/acrylic acid), poly(ethylene glycol) bis(2-aminoethyl), poly (ethylene glycol) monomethyl ether, poly(ethylene glycol)bisphenol A diglycidyl ether adduct, poly(ethylene oxide-bpropylene oxide), poly(ethylene/acrylic acid) 92:8, poly(llysine hydrobromide), poly(l-lysine hydrobromide), poly (maleic acid), poly(n-butyl acrylate/2methacryloxyethyltrimethylammonium bromide), poly(Niso-propylacrylamide), poly(N-vinylpyrrolidone/2dimethylaminoethyl methacrylate), dimethyl sulfatequaternary, poly(N-vinylpyrrolidone/vinyl acetate), poly(oxyethylene) sorbitan monolaurate (Tween 20®), poly (styrenesulfonic acid), poly(vinyl alcohol), N-methyl-4(4′formylstyryl)pyridinium, methosulfate acetal, poly(vinyl methyl ether), poly(vinylamine) hydrochloride, poly(vinylphosphonic acid), poly(vinylsulfonic acid) sodium salt, and polyaniline.

Regardless of the material, the tie layer 335 may or may not be rotational spun. Further, in certain embodiments the stent 302 may include two or more tie layers. The tie layer 335 may be formed in any manner known in the art and attached to the inner 325 and outer 330 layers in any manner known in the art. For example, the tie layer 335 may comprise a sheet of material which is wrapped around the inner layer 325 or a tube of material which is slipped over the inner layer 325 which is then heat shrunk or otherwise bonded to the inner 325 and outer 330 layers. Further, in embodiments where the tie layer is rotational spun, it may be rotational spun directly onto the inner layer 325, the scaffolding, or both. In some instances the tie layer 335 may be melted after the stent 302 is constructed to bond the tie layer 335 to adjacent layers of the stent covering.

Furthermore, tie layers may be configured to change the overall properties of the medical appliance. For example, in some instances a cover or construct comprised solely of rotational spun PTFE (of the desired pore size) may not have desired tensile or burst strength. A tie layer comprised of a relatively stronger material may be used to reinforce the PTFE inner layer, the PTFE outer layer, or both. For example, in some instances FEP layers may be used to increase the material strength of the cover. Again, as discussed above, the tie layer may also be configured as a portion of the construct configured to be impervious to tissue ingrowth or migration.

Further, one or more layers of rotational spun PTFE may be used in connection with a scaffolding structure other than that shown herein. In other words, the disclosure above relating to covers, layers, tie layers, and related components is applicable to any type of scaffolding structure as well as to stents or grafts with no separate scaffolding structure at all.

FIGS. 4A-4E illustrate certain steps in a process of manufacturing a multilayer construct for use in connection with a medical appliance. More specifically, these figures illustrate a process of creating a stent covered with rotational spun material. Again, this disclosure is equally relevant to all medical appliances which may comprise a cover or multilayered construct, including grafts, patches, stents, and so on. Additionally, as suggested in the additional examples disclosed below, the illustrated steps may be optional in some instances or augmented by additional steps in others.

FIG. 4A illustrates a covering inner layer 425 disposed around a mandrel 416. As described above, the inner layer 425 may be rotational spun directly onto the mandrel 416, including instances wherein the mandrel 416 was rotating during the process. In the illustrated embodiment, the inner layer 425 can be rotational spun onto a rotating mandrel 416 such that the resultant tube of material has no seam. After the inner layer 425 is rotational spun onto the mandrel 416, the inner layer 425 may then be sintered. In the case of PTFE, the membrane may be sintered at temperatures of about 385 degrees C, including temperatures from about 360 degrees C to about 400 degrees C. Sintering may tend to set the structure of the PTFE, meaning sintering reduces the softness or flowability of the PTFE. Furthermore, as discussed above, sintering or otherwise heat treating the mat may evaporate any water or PEO mixed with the PTFE, resulting in a material comprised substantially of pure PTFE.

Once the inner layer 425 is sintered, the tube of material may be removed from the mandrel 416, as illustrated in FIG. 4B. As shown in the illustrated embodiment, the inner layer 425 may be “peeled” from the mandrel 416 to initially break any adherence of the inner layer 425 to the mandrel 416. The inner layer 425 may also be removed by pushing the covering with respect to the mandrel 416, causing the material to bunch as it is removed from the mandrel 416. In some embodiments, low friction coatings may alternatively or additionally be applied to the mandrel 416 before the inner layer 425 is rotational spun. The inner layer 425 may then be reapplied to the mandrel 416, by slipping the inner layer 425 over the mandrel 416, as illustrated in FIG. 4C.

Once the inner layer 425 is reapplied to the mandrel 416, a wire scaffolding 420 can be formed over the mandrel 416 and the inner layer 425, as shown in FIG. 4D. FIG. 4E illustrates an outer layer 430 of material which may then be rotational spun onto the scaffolding 420 and the inner layer 425. The entire construct may then be sintered. Additional layers may also be added through similar processes.

Many variations to the above-described process are within the scope of the present disclosure. For example, one or more layers may be applied by wrapping strips or mats of material around the mandrel 416 and/or the other layers. Further, some of the layers may be applied by spray or dip coating the mandrel 416 and/or the other layers. It is within the scope of this disclosure to vary the process above to apply to any of the layers, or any additional layers, using any method disclosed herein.

In another example, a stent may be comprised of an inner layer of rotational spun PTFE, a tie layer of FEP, and an outer layer of PTFE. The properties of each of these layers, including percent porosity, mat thickness, fiber diameter, and/or average pore size may be controlled to form a covering layer that inhibits the growth of tissue into or through a particular layer or that permits endothelial growth or attachment on a particular layer.

In some such embodiments, the inner layer of PTFE may be spun on a mandrel, sintered, removed from the mandrel, and replaced on the mandrel then a scaffolding structure applied around the inner layer (analogous to the procedure illustrated in FIGS. 4A-4D). The FEP tie layer may then be applied by dipping, spraying, applying a film layer, electrospinning, rotational spinning, extrusion, or other processing.

In some embodiments, the FEP layer may be heated such that the FEP becomes soft, in some cases flowing into open spaces in adjacent PTFE layers. This may tie the FEP layer to adjacent PTFE layers. In some instances, heating the construct to about 325 degrees C may allow the FEP to partially flow into openings in adjacent PTFE layers, without the FEP completely flowing through the PTFE mat.

In another particular example, an inner layer of PTFE may be rotational spun on a mandrel, sintered, removed, and replaced, then a scaffolding structure applied around the inner layer. An FEP tie layer may then be applied as a film layer. In some instances this tie layer may be “tacked” into place, for example, by a soldering iron. A tube of PTFE (which may be formed separately by rotational spinning onto a mandrel and sintering) may then be disposed over the FEP film layer. The entire construct may then be pressured, for example, by applying a compression wrap. In some embodiments this wrap may comprise any suitable material, including a PTFE-based material. In other embodiments a Kapton film may be wrapped around the construct before the compression wrap, to prevent the construct from adhering to the compression wrap.

The compressed layers may then be heated above the melting temperature of the FEP tie layer, but below the sintering temperature of the PTFE. For example, the melt temperature of the FEP may be from about 264 degrees C to about 380 degrees C, including about 325 degrees C. PTFE may be sintered at temperatures from about 360 degrees C to about 400 degrees C. Thus, the entire construct may be heated to an appropriate temperature such as about 325 degrees C. In some embodiments the construct may be held at this temperature for about 15 to about 20 minutes. Heating the FEP layer to about 325 degrees C may allow the FEP layer to remain substantially impervious to tissue ingrowth and/or attachment, creating a “barrier” layer within the construct, while still adhering the FEP to adjacent layers of PTFE. In other embodiments, heating the construct to higher temperatures, such as about 350 degrees C or more may be configured to allow the FEP to flow around the PTFE such that the entire construct has a higher degree of porosity and the FEP layer is not as impervious to ingrowth.

The joining of the FEP tie layer to the PTFE outer and inner cover layers may increase the strength of the finished covering. The construct may then be cooled and the compression wrap and the Kapton film discarded. The construct may then be removed from the mandrel.

A stent formed by the exemplary process described above may be configured with desired characteristics of porosity and strength. In some instances the FEP material may coat the PTFE nanofibers but still allow for sufficient porosity to permit tissue ingrowth and/or endothelial attachment or growth. The degree to which the FEP coats the PTFE may be controlled by the temperature and time of processing. The lower the temperature and/or the shorter the time the construct is held at temperature, the less the FEP may flow. In some instances a tie layer of FEP which is impervious to tissue ingrowth into or through the layer may be formed by heating the construction only to about 270 degrees C.

In some embodiments the PTFE outer and/or inner cover layer may be produced by using rotational spun mats. Some mats produced by rotational spinning may have parameters, such as thickness, which vary along one or more dimension of the mat. For instance, this method can involve creating mats where the thickness varies along a first dimension of the mat, such as the length or width. Variability in thickness throughout the mat can be controlled during the spinning process, allowing for management of the mat's uniformity or isotropic characteristics.

In some embodiments the PTFE outer and/or inner cover layer may be produced by using rotational spun mats with a thickness from about 20 micrometers to about 120 micrometers, from about 30 micrometers to about 110 micrometers, or from about 40 micrometers to about 100 micrometers. More specifically, in some cases, an average thickness of the mat can be anywhere within the range from about 20 micrometers to about 120 micrometers, from about 30 micrometers to about 110 micrometers, or from about 40 micrometers to about 100 micrometers. In some embodiments the PTFE outer and/or inner cover layer may be produced by using rotational spun mats with a thickness from about 30 micrometers to about 300 micrometers. In some cases, the PTFE outer layer and/or inner cover layer may be produced by using rotational spun mats with a thickness from about 30 micrometers to about 200 micrometers.

In embodiments, the thickness of the mat can vary no more than 100 micrometers, or 80 micrometers, or 60 micrometers, or 40 micrometers, or 20 micrometers, along a first dimension of the mat (e.g., length or width). Otherwise stated, in embodiments, a maximum thickness of the mat can be no more than 100 micrometers, 80 micrometers, or 60 micrometers, or 40 micrometers, or 20 micrometers, more than a minimum thickness of the mat.

In embodiments, a maximum thickness of the mat can be no more than 180%, 160%, or 140% of an average thickness of the mat. A minimum thickness of the mat can be no less than 60%, 40%, or 20% of an average thickness of the mat.

In some embodiments, a standard deviation of the mat thickness can be no more than 60 micrometers, 40 micrometers, or 20 micrometers, or 15 micrometers, or 10 micrometers.

FIG. 5 illustrates a stent 502 which comprises a scaffolding structure 520 and a covering 524. The covering 524 may be comprised of any combination of layers disclosed herein. Additionally, the stent 502 of FIG. 5 includes a cuff 540 at both ends of the stent 502. In other embodiments a cuff 540 may only be located at one end of the stent 502.

The cuff 540 may comprise an additional covering layer on the outside diameter of the stent 502, disposed adjacent to one or both ends of the stent 502. The cuff 540 may be configured to promote tissue ingrowth, attachment, and/or incorporation into the cuff 540; for example the cuff 540 may be more porous than an outer layer of the covering 524 of the stent 502. Factors such as porosity, type of covering or coating, type of material, use of organic material, and/or use or composite materials formed of synthetic material and organic material may be used to create a cuff 540 configured for tissue ingrowth. Again, the cuff 540 may be configured to promote tissue ingrowth and/or the growth or attachment of endothelial cells at one or both ends of the stent 502. When implanted in the body, the cuffs 540 may tend to anchor the ends of the stent 502 with respect to the vessel walls, reducing the relative movement of the stent ends with respect to the vessel walls. Such a reduction in movement may lessen irritation of the vessel by the stent ends, minimizing complications such as stenosis. Cuffs 540 may be configured for use in CVO type applications in some instances. Furthermore, a band of porous material analogous to the stent cuff 540 illustrated may be coupled to any medical appliance to anchor a portion of such a device.

In some embodiments, the outer layer of the covering 524 of the stent 502 may be relatively non-porous to inhibit tissue ingrowth into or through the outer layer, but the cuff 540, disposed about the outer layer, may provide a section near each end at which some tissue ingrowth, attachment, or incorporation may occur.

The cuff 540 may be comprised of a rotational spun material, such as PTFE, and may be bonded to the outer covering layer through any method, including methods of multilayer device construction described herein. For example, a layer of FEP may be disposed between the outer covering layer and the cuff 540 and heated to bond the layers. In other embodiments the cuff 540 may comprise a collagen layer which is coupled to the stent. Further, a co-rotational spun collagen and PTFE cuff 540 may be utilized.

As discussed above, a medical appliance may be a stent that comprises a scaffolding structure and a covering. The covering may include an inner layer, an outer layer, and a tie layer. The medical appliance may be fabricated so that the medical appliance may be crimped into a low-profile. In some embodiments, the inner layer may be fabricated so that the inner layer comprises a uniform thickness enabling the medical appliance to have an even smaller low-profile compared to a medical appliance with a non-uniform thickness, enabling the medical appliance to fit within smaller French catheters for delivery.

As discussed above, the formation of the inner layer creates a tube of material without a seam. The inner layer may be referred to as a base tube for the medical appliance. The scaffolding, the tie layer, and the outer layer may be added to the base tube. In some embodiments, the base tube comprises a single layer. In some embodiments, the base tube comprises more than one layer.

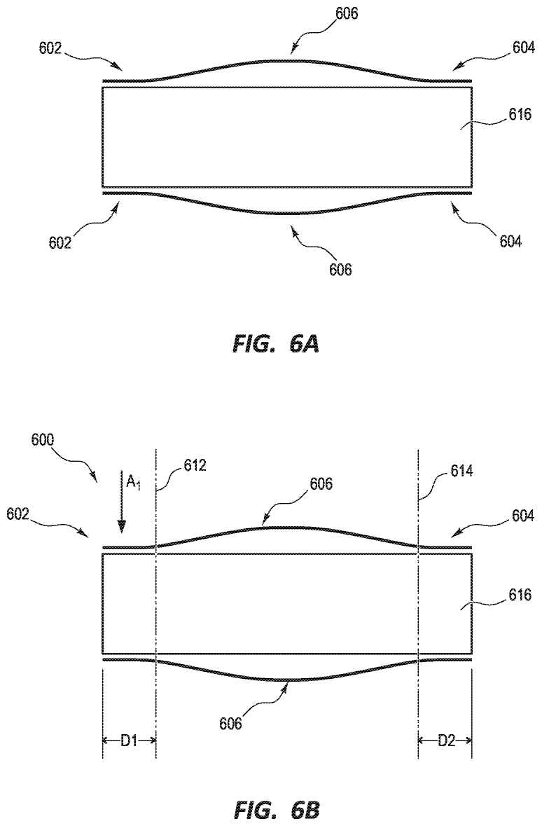

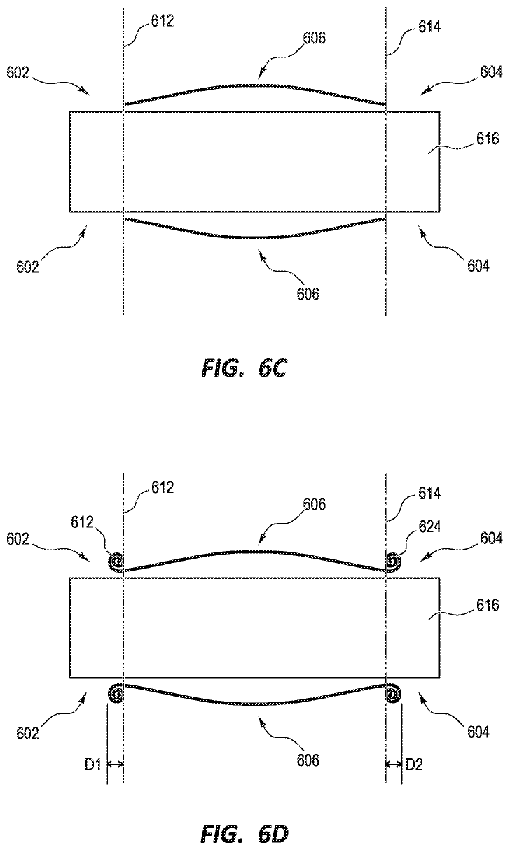

FIGS. 6A-6D describe a process of obtaining a base tube 600 with a uniform thickness for a medical appliance that will enable the medical appliance to have a low-profile.

In some embodiments the base tube 600 may have a percent porosity of about 40% to about 80%, including about 50%; a thickness of about 20 micrometers to about 100 micrometers, including from about 40 micrometers to about 80 micrometers; and fiber diameters from about 50 nanometers to about 3 micrometers.

In some embodiments the base tube 600 may be produced by serially depositing polymeric fibers on a mandrel, such as mandrel 616. Serially depositing fibers may include rotational spinning, electrospinning, and the like. Some mats produced by rotational spinning may have parameters, such as thickness, which vary along one or more dimension of the mat. For instance, this can involve creating mats where the thickness varies along a first dimension of the mat, such as the length or width. Variability in thickness throughout the mat can be controlled during the spinning process, allowing for the management of the mat's uniformity or isotropic characteristics.

In some embodiments the base tube 600 may be produced by using rotational spun mats with a range of predetermined thickness. The range of predetermined thickness may be from about 20 micrometers to about 120 micrometers, from about 30 micrometers to about 110 micrometers, or from about 40 micrometers to about 100 micrometers. More specifically, in some cases, an average thickness of the base tube 600 can be anywhere within the range from about 20 micrometers to about 120 micrometers, from about 30 micrometers to about 110 micrometers, or from about 40 micrometers to about 100 micrometers. The range of predetermined thickness may be from about 30 micrometers to about 300 micrometers. In some cases, the range of predetermined thickness may be from about 30 micrometers to about 200 micrometers.

In embodiments, the range of predetermined thickness of the base tube 600 can vary no more than 100 micrometers, or 80 micrometers, or 60 micrometers, or 40 micrometers, or 20 micrometers, along a first dimension of the base tube 600 (e.g., length or width). Otherwise stated, in embodiments, a maximum thickness of the base tube 600 can be no more than 100 micrometers, 80 micrometers, or 60 micrometers, or 40 micrometers, or 20 micrometers, more than a minimum thickness of the base tube 600.

In embodiments, a maximum thickness of the base tube 600 can be no more than 180%, 160%, or 140% of an average thickness of the base tube 600. A minimum thickness of the base tube 600 can be no less than 60%, 40%, or 20% of an average thickness of the base tube 600.

In some embodiments, a standard deviation of the thickness of the base tube 600 can be no more than 60 micrometers, 40 micrometers, or 20 micrometers, or 15 micrometers, or 10 micrometers.

Adjusting the various parameters during serially depositing the polymer fibers on the mandrel 616 may affect the thickness of the base tube 600 and may enable a uniform thickness while serially depositing the polymeric fibers on the mandrel 616.

For example, as discussed in regard to FIG. 2A, the polymeric fibers may be serially deposited on vertical mandrels, such as mandrel 216. In some embodiments, polymeric fibers may be serially deposited on horizontal mandrels as horizontal mandrels may be able to produce a base tube 600 with relatively uniform thickness around the mandrel 616. In other words, the thickness of the base tube 600 is within the range of predetermined thickness.

In some embodiments, the polymeric fibers may be serially deposited on mandrels 616 that rotate. In some embodiments, the polymeric fibers may be serially deposited on mandrels 616 that are conveyed on a conveyer system to produce a base tube 600 with a more uniform thickness. In other words, the thickness of the base tube 600 is within the range of predetermined thickness. For example, as illustrated in FIG. 2A, the mandrels 216 may be conveyed in a circular motion around the spinneret 210.

In some embodiments, the polymeric fibers may be serially deposited on mandrels 616 that are rotated and conveyed on a conveyer system to produce a base tube 600 with a more uniform thickness. In other words, the thickness of the base tube 600 is within the range of predetermined thickness.

In some embodiments, polymeric fibers may be serially deposited on the mandrel 616 in the presence of air currents. The air currents may be able to deliver the polymeric fibers more uniformly along the length of the mandrel 616 to produce a base tube 600 with a more uniform thickness. The air currents may be produced by fans to either push or draw the fibers in a direction toward the mandrel 616. In other words, the thickness of the base tube 600 is within the range of predetermined thickness.

In some embodiments, the mandrels 616 may comprise a vacuum that draws the polymeric fibers toward the mandrels 616 to produce a base tube 600 with a more uniform thickness. In other words, the thickness of the base tube 600 is within the range of predetermined thickness.

In some embodiments, the placement of the spinnerets relative to the mandrels 616 may affects the thickness and uniformity of the polymeric fibers on the mandrels 616. Adjusting the distance between the spinnerets and the mandrels 616 may produce a base tube 600 with a more uniform thickness. In other words, the thickness of the base tube 600 is within the range of predetermined thickness.

In some embodiments, less polymeric fibers may be serially deposited on the mandrel 616 for the base tube 600 than the number of polymeric fibers that are serially deposited on an outer layer of the medical appliance. The fewer number of polymer fibers may produce a base tube 600 with a more uniform thickness. In other words, the thickness of the base tube 600 is within the range of predetermined thickness.

FIG. 6A illustrates a cross-sectional view of a base tube 600 for a low-profile medical appliance. FIG. 6A illustrates the base tube 600 disposed around a mandrel 616 after a plurality of polymeric fibers are serially deposited on the mandrel 616. As described above, the base tube 600 may be rotational spun directly onto the mandrel 616, including instances wherein the mandrel 616 was rotating during the process. In the illustrated embodiment, the base tube 600 can be rotational spun onto a rotating mandrel 616 such that the resultant tube of material has no seam. In some embodiments, the base tube 600 may be electrospun onto the mandrel 616.

The cross-sectional view of the base tube 600 illustrates the thickness of the base tube 600. As shown in FIG. 6A, the thickness of the base tube 600 is not entirely uniform. Typically during the serially depositing process, the thickness of a first end 602 and a second end 604 (opposite the first end 602) of the base tube 600 are not as thick as a central portion 606 of the base tube 600. In other words, the thickness of the base tube 600 increases from the first end 602 to the central portion 606 and decreases from the central portion 606 to the second end 604.

However, other thickness profiles other than the one illustrated in FIG. 6A may be produced. For example, a thickness profile in which the thickest portion is disposed at the first end 602 and the thinnest portion is disposed at the second end 604. Another example may be the inverse of FIG. 6A in which the thickest portions are at the first end 602 and the second end 604 and the central portion 606 is thinner than the first end 602 and the second end 604. The disclosure below of manipulating the base tube 600 so that the entire base tube 600 is within the range of predetermined thickness, such as discarding portions outside the range of predetermined thickness (e.g., too thick or too thin) or compressing thicker portions, applies other thickness profiles as well.

After the base tube 600 is formed by serially depositing the polymeric fibers onto the mandrel 616, the base tube 600 may be validated to determine if the thickness of the base tube 600 is within the range of predetermined thickness. For example, validation may be performed by measuring the thickness of the base tube 600, which is discussed in more detail below. In addition, the base tube 600 may be validated by weighing the base tube 600 to determine its weight. If the base tube 600 is within a range of predetermined weight, the base tube 600 may be validated as comprising a thickness with the range of predetermined thickness.

The weight of the base tube 600 is a function of the diameter and the length of the base tube 600. Through validation testing, the Applicant has determined that if the base tube 600 has a weight within the range of predetermined weight, then the base tube 600 comprises a thickness with the range of predetermined thickness. The weight varies based on the diameter and the overall length of the base tube 600. For example, a range of predetermine weight is based on the diameter and the length of the base tube. For example, the fabricated base tube is measured for its length and diameter. After the length and diameter are determined, the range of predetermined weight is determined. The fabricated base tube is weighed and if the fabricated base tube is within the range of predetermined weight, the base tube is validated.

In another process of validation, after the base tube 600 is formed by serially depositing the polymer fibers on the mandrel 616, the thickness of the base tube 600 may be identified. Identifying the thickness of the base tube 600 may be performed using a laser micrometer or another suitable device. As illustrated in FIG. 6B, the laser micrometer may measure the thickness of the base tube 600. Arrow A₁ represents the laser of the laser micrometer measuring the thickness of the base tube 600. For example, the laser micrometer may measure the thickness of the base tube 600 at the first end 602 along the entire length of the base tube to the second end 604.

In some embodiments, the laser micrometer may measure the base tube 600 from the first end 602 inward until the thickness of the base tube 600 reaches a first point 612 in which the thickness of the base tube 600 is within the range of predetermined thickness. Similarly, the lase micrometer may measure the thickness of the base tube 600 from the second end 604 inward until the thickness of the base tube 600 reaches a second point 614 in which the thickness of the base tube 600 is within the range of predetermined thickness.

In the illustrated embodiment, the length of the first end 602 to the first point 612 is equal to the length of the second end 604 to the second point 614. However, in many instances this will not be the case. In some embodiments, the length of the first end 602 to the first point 612 is longer than the length of the second end 604 to the second point 614. In some embodiments, the length of the first end 602 to the first point 612 is shorter than the length of the second end 604 to the second point 614. These discrepancies in length may be explained via the parameters for serially depositing the polymeric fibers on the mandrel 616.

Once first point 612 and second point 614 are determined, the rest of the base tube 600 between the first point 612 and the second point 614 is determined to be within the range of predetermined thickness. Through validation testing and SEM imagining, the Applicant has determined that the thickness of the base tube 600 between the first point 612 and the second point 614 is within the range of predetermined thickness. Accordingly, after the first point 614 and the second point 614 are determined, the base tube 600 may be manipulated so that the entire length of the base tube 600 has a thickness that is within the range of predetermined thickness.

In some embodiments, manipulating of the base tube 600 may include discarding the portions of the base tube 600 that are outside the range of predetermined thickness. For example, the first end 602 up to the first point 612 and the second end 604 up to the second point 614 are discarded, e.g., discarded portions D1 and D2. The discarded portions D1 and D2 may be removed while the base tube 600 is disposed on the mandrel 616 or may be removed after the base tube 600 is removed from the mandrel 616. FIG. 6C illustrates a cross-sectional view of the base tube 600 on the mandrel 616 with the discarded portions D1 and D2 removed with only portions of the base tube 600 within the range of predetermined thickness remaining. The discarded portions D1 and D2 may be discarded before or after sintering of the base tube 600.

As illustrated in FIG. 6D, in some embodiments, manipulating the base tube 600 may include bunching up the first end 602 up to the first point 612 to form a first ridge 622 and bunching up the second end 604 up to the first point 614 to form a second ridge 624. Bunching may be performed by rolling the first end 602 inward until it reaches the first point 612 and rolling the second end 604 inward until it reaches the second point 614, The first ridge 622 and the second ridge 624 form markers that indicate what portions of the base tube 600 should be discarded. Once the first ridge 622 and the second ridge 624 are formed, these portions are discarded, e.g., discarded portions D1 and D2. The discarded portions D1 and D2 may be removed while the base tube 600 is disposed on the mandrel 616 or may be removed after the base tube 600 is removed from the mandrel 616. Once the discarded portions D1 and D2 are removed from the base tube 600, it appears similar to the base tube 600 illustrated in FIG. 6C, which illustrates a cross-sectional view of the base tube 600 on the mandrel 616 with the discarded portions D1 and D2 removed with only portions of the base tube 600 within the range of predetermined thickness remaining. The discarded portion D1 and D2 may be discarded before or after sintering of the base tube 600.

In some embodiments, the laser micrometer may measure the base tube 600 and the thickness of the base tube 600 is greater than the range of predetermined thickness. Accordingly, the base tube 600 may be manipulated so that the entire length of the base tube 600 has a thickness that is within the range of predetermined thickness. For example, the base tube 600 may be compressed to flatten the thickness of the base tube 600 so that the entire length of the base tube 600 is within the range of predetermined thickness.

After the base tube 600 is serially deposited (e.g., rotational spun) onto the mandrel 616, the base tube 600 may then be sintered. In the case of PTFE, the membrane may be sintered at temperatures of about 385 degrees C, including temperatures from about 360 degrees C to about 400 degrees C. Sintering may tend to set the structure of the PTFE, meaning sintering reduces the softness or flowability of the PTFE. Furthermore, as discussed above, sintering or otherwise heat treating the mat may evaporate any water or PEO mixed with the PTFE, resulting in a material comprised substantially of pure PTFE.

Once the base tube 600 is sintered, the base tube 600 may be removed from the mandrel 416, similar to FIG. 4B. The base tube 600 may be “peeled” from the mandrel 616 to initially break any adherence of the base tube 600 to the mandrel 616. The base tube 600 may also be removed by pushing the base tube 600 with respect to the mandrel 616, causing the material to bunch as it is removed from the mandrel 616. In some embodiments, low friction coatings may alternatively or additionally be applied to the mandrel 616 before the base tube 600 is serially deposited. The base tube 600 may then be reapplied to the mandrel 616, by slipping the base tube 600 over the mandrel 516, similar to FIG. 4C.

Once the base tube 600 is reapplied to the mandrel 616, a wire scaffolding can be formed over the mandrel 616 and the base tube 600, similar to FIG. 4D. An outer layer of material which may then be serially deposited onto the scaffolding and the base tube 600. The entire construct may then be sintered. Additional layers may also be added through similar processes.

For example, in some embodiments, the tie layer be disposed above or below the wire scaffolding. In some embodiments, the wire scaffolding may be disposed within the tie layer.

Many variations to the above-described process are within the scope of the present disclosure. For example, one or more layers may be applied by wrapping strips or mats of material around the mandrel 616 and/or the other layers. Further, some of the layers may be applied by spray or dip coating the mandrel 616 and/or the other layers. It is within the scope of this disclosure to vary the process above to apply to any of the layers, or any additional layers, using any method disclosed herein.

The current disclosure relates to medical appliances, including stents, which may comprise a frame structure provided in connection with one or more coverings or coatings. It will be appreciated that, though particular structures, coverings, and coatings are described herein, any feature of the frames or coverings and/or coatings described herein may be combined with any other disclosed feature without departing from the scope of the current disclosure. Further, as used herein, the term “frame” refers to a support structure for use in connection with a medical appliance. For instance, a scaffolding structure, such as that described in connection with FIGS. 4A-E, above, is an example of a frame used in connection with a medical appliance. In some embodiments, a medical appliance-such as a stent-may comprise frame alone, with no covering, coating, or other components.

Moreover, the current disclosure is applicable to a wide variety of medical appliances which may utilize any of the rotational spun mats disclosed herein, including medical appliances which comprise multilayered constructs. For example, a hernia patch may comprise a two layered construction, which one side of the patch configured to allow tissue ingrowth and/or attachment (for bonding and healing) and the other side configured to resist such ingrowth and/or attachment (to make the second side “slippery” with respect to surrounding tissue. Further, a patch as described above may also comprise a tie layer disposed between the two exterior layers. The tie layer may be configured resist tissue ingrowth or attachment into or through the patch and/or to provide mechanical properties such as strength to the construct.

Frames for use in connection with medical appliances may be fabricated or formed into particular geometries through a variety of means. For example, a frame may be cut from a single tube of material, including embodiments wherein the frame is first laser cut, then expanded. In other embodiments, the frame may be molded, including embodiments wherein the frame is molded from a polymeric material. In still other embodiments, powder metallurgical processes, such as powdered compression molding or direct metal laser sintering, may be used. Additionally, in some embodiments, any covering as disclosed herein may be applied to any type of frame, for example, laser cut frames, polymeric frames, wire frames, and so forth.

A frame may be designed such that the midsection is “harder” than the ends. The “hardness” of the frame refers to the relative strength of the structure (e.g., its compressibility). A harder portion of the frame will have greater strength (i.e., exert a greater radial outward force) than a softer portion. In one embodiment, the midsection is harder than the proximal and distal end sections which are relatively softer. Further, a frame may be configured to be flexible to facilitate the ability of the device to conform to the native anatomy at which the device is configured for use. Similarly, covered devices may be configured with covers which conform to the native anatomy at a therapy site.

Additionally, the frame may be configured to allow the entire device to be crimped into a relatively low-profile configuration for delivery. For example, devices of a certain diameter or constrained profile are more feasible for delivery at certain vascular or other access points than others. For example, in many instances a device configured for insertion via the radial artery may be relatively smaller than devices configured for insertion via the generally larger femoral artery. A frame may be configured to be crimped into a particular profile to enable potential access at various or desired access points. Similarly, devices having no frame may be configured to be disposed in a particular profile to facilitate access and delivery. Once a device is positioned within the body it may be expanded or deployed in a number of ways, including use of self-expanding materials and configurations. Additionally, some configurations may be designed for expansion by a secondary device, such as a balloon.

The overall frame design may be configured to optimize desired radial force, crush profile, and strain profile. The frame design parameters may each be configured and tuned to create desired characteristics. For example, the strain profile may be configured to be less than the failure point for the material being used.

As will be apparent to those skilled in the art having the benefit of this disclosure, frames with a hard midbody and soft ends may be desirable for a variety of applications. Further, in some instances a basically “symmetric” frame may be desirable; in other words, a frame with certain properties at the midbody section and other properties at the ends, where the properties at both ends are substantially identical. Of course, other embodiments may have varied properties along the entire length of the frame.

In some instances, a radiopaque marker may be crimped around the stent in such a manner as to couple the end of the frame. Additionally other methods known in the art may be utilized.

Furthermore, in some embodiments the frame may be configured with radiopaque markers at one or more points along the frame. Such markers may be crimped to the frame. In other embodiments a radiopaque ribbon, for example a gold ribbon, may be threaded or applied to the frame. In some embodiments these markers may be located at or adjacent to one or both ends of the frame. Any radiopaque material may be used, for example gold or tantalum. Radiopaque elements may be configured to facilitate the delivery and placement of a device and/or to facilitate viewing of the device under fluoroscopy.

It will be appreciated that in certain embodiments a frame may have a flare at both the proximal and distal ends, only at the proximal end or only at the distal end, or at neither end. In certain of these embodiments the frame may have a substantially constant diameter in the midbody zone of the frame, with the ends flaring outward to a larger diameter. It will be appreciated that the geometry of the flares at the proximal and distal ends may or may not be the same. The frame may or may not be configured with flared ends regardless of the midbody diameter employed.

A frame may be formed from a single continuous wire. In some embodiments the wire may be comprised of Nitinol (ASTM F2063), or other suitable materials.

In some embodiments, a stent, graft, or other tubular device may comprise a tapered segment along the length of the device. A taper may be configured to reduce the velocity of fluid flow within the device as the fluid transitions from a smaller diameter portion of the device to a larger diameter portion of the device. Reducing the fluid velocity may be configured to promote laminar flow, including instances wherein a tubular member is tapered to promote laminar flow at the downstream end of the device.

Further, in some embodiments, a stent or other tubular member may be positioned at a junction between two or more body lumens.

Use of rotational spun coatings may facilitate application of a covering of uniform thickness along a tapered stent. For example, in some embodiments, rotational spun coatings may be configured to evenly coat devices comprised of various geometries. A rotational spun coating may deposit a substantially even coating along various geometries such as tapers, shoulders, and so forth.

While specific embodiments of stents and other medical appliances have been illustrated and described, it is to be understood that the disclosure provided is not limited to the precise configuration and components disclosed. Various modifications, changes, and variations apparent to those of skill in the art having the benefit of this disclosure may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not as a limitation of the scope of the present disclosure in any way. It will be apparent to those having skill in the art, and having the benefit of this disclosure, that changes may be made to the details of the above-described embodiments without departing from the underlying principles of the disclosure herein.

1. A medical appliance comprising:
a first layer of rotational spun polytetrafluoroethylene (PTFE) disposed on an inside surface of the medical appliance;
a tie layer coupled to the first layer of rotational spun PTFE, wherein the tie layer is impervious to cell migration into the tie layer; and
a second layer of PTFE,
wherein the tie layer is disposed between the first layer of rotational spun PTFE and the second layer of PTFE,
wherein the first layer of rotational spun PTFE of a thickness ranging from about 30 micrometers to about 300 micrometers and varying no more than about 100 micrometers.

2. The medical appliance of claim 1, wherein an average of the thickness of the first layer of rotational spun PTFE is within a range from about 30 micrometers to about 200 micrometers.

3. The medical appliance of claim 1, wherein the thickness of the first layer of rotational spun PTFE varies no more than 80 micrometers throughout the first layer of rotational spun PTFE.

4. The medical appliance of claim 1, wherein a maximum thickness of the first layer of rotational spun PTFE is no more than 180% of an average thickness of the first layer of rotational spun PTFE, wherein a minimum thickness of the first layer of rotational spun PTFE is no less than 20% of an average thickness of the first layer of rotational spun PTFE.

5. The medical appliance of claim 1, wherein a standard deviation of the thickness of the first layer of rotational spun PTFE is no more than 60 micrometers.

6. The medical appliance of claim 1, wherein the second layer of PTFE comprises expanded PTFE (ePTFE).

7. A method of forming a uniform base tube for a low-profile medical appliance comprising:
serial depositing polymeric fibers on a mandrel to form a base tube for the low-profile medical appliance;
identifying a thickness of the base tube to determine if the thickness of an entire length of the base tube is within a range of predetermined thickness; and
manipulating the base tube so that the thickness of the entire length of the base tube is within the range of predetermined thickness.

8. The method of claim 7, wherein the range of predetermined thickness ranges from about 30 micrometers to about 300 micrometers and varies no more than about 100 micrometers along the entire length of the base tube.

9. The method of claim 7, further comprising adjusting parameters of the serial depositing so that the thickness of the base tube is within the range of predetermined thickness.

10. The method of claim 9, wherein adjusting the parameters of the serial depositing includes introducing air currents during the serial depositing so that the thickness of the base tube is within the range of predetermined thickness.

11. The method of claim 9, wherein adjusting the parameters of the serial depositing includes conveying the mandrel during the serial depositing so that the thickness of the base tube is within the range of predetermined thickness.

12. The method of claim 7, wherein manipulating the base tube comprises compressing the base tube so that the base tube is within the range of predetermined thickness.

13. The method of claim 7, wherein identifying the thickness of the base tube comprises measuring the thickness of a first end of the base tube inward until a first point where the thickness of the base tube is within the range of predetermined thickness and measuring the thickness of a second end, opposite the first end, of the base tube inward until a second point where the thickness of the base tube is within the range of predetermined thickness.

14. The method of claim 13, wherein manipulating the base tube comprises discarding the first end up to the first point and the second end of the base tube up to the second point that are outside the range of predetermined thickness so that the base tube is within the range of predetermined thickness.

15. The method of claim 13, wherein manipulating the base tube comprises bunching up the first end outside the range of predetermined thickness up to the first point forming a first ridge; and
bunching up the second outside the range of predetermined thickness up to the second point forming a second ridge.

16. The method of claim 15, wherein manipulating the base tube comprises discarding the first end up to the first ridge and the second end up to the second ridge so that the base tube is within the range of predetermined thickness.

17. A method of forming a base tube for a low-profile medical appliance comprising:
spinning a plurality of fibers on a mandrel to form a base tube for the low-profile medical appliance; and
validating a thickness of the base tube is within a range of predetermined thickness by weighing the base tube,
wherein validation of the thickness of the base tube is determined by a weight of the base tube as a function of the diameter and length of the base tube.

18. The method of claim 17, further comprising:
identifying the thickness of the base tube to determine if the thickness of an entire length of the base tube is within a range of predetermined thickness; and
manipulating the base tube so that the thickness of the entire length of the base tube is within the range of predetermined thickness.
